(19) United States
(12) Patent Application Publication
(10) Pub. No.: US 2012/0289638 A1
(43) Pub. Date: Nov. 15, 2012

(54) POLYBUTADIENE DERIVATIVE COMPOSITION

(75) Inventors: Hiroki Hashimoto, Ichihara-shi (JP); Yukikazu Nobuhara, Ichihara-shi (JP)

(73) Assignee: NIPPON SODA CO., LTD., Tokyo (JP)

(21) Appl. No.: 13/520,940

(22) PCT Filed: Jan. 13, 2011
(86) PCT No.: PCT/JP2011/000143

§ 371 Date: Jul. 6, 2012

(30) Foreign Application Priority Data
Jan. 15, 2010 (JP) 2010-007273

(51) Int. Cl. C08K 5/37; C07C 69/587

(52) U.S. Cl. 524/384; 560/225

(57) ABSTRACT

An object of the present invention is to provide a composition containing a stabilizer which does not turn yellow upon curing and which acts as a polymerization inhibitor during production and storage of a (meth)acrylic modified (hydrogenated) polybutadiene. The curable composition is characterized in that it contains 0.1-99.9999% by weight of component (A) which is a (meth)acrylic modified (hydrogenated) polybutadiene, and 0.0001-10% by weight of component (B) which is a compound represented by formula (IV).

### TECHNICAL FIELD

The present invention relates to a curable composition and a cured product thereof, wherein the curable composition contains a (meth)acrylic modified (hydrogenated) polybutadiene which is a colorless transparent cured product suitable as an optical material.

The present application claims priority from Japanese Patent Application No. 2010-7273 filed on Jan. 15, 2010, the content of which is incorporated herein.

### BACKGROUND ART

When liquid polybutadiene is cured, it results in a resin having superior water and moisture resistance, chemical resistance, electric properties (high dielectric strength, low dielectric constant, arc resistance) and transparency as well as being a highly tough substance. Thus, such a resin has been conventionally used for various purposes.

Further, there has been known a liquid polybutadiene in which a (meth)acrylic group is introduced at the terminal in order to improve curing property of the liquid butadienes (Patent Documents 1 and 2). These compounds, when added a stabilizer, inhibited a (meth)acrylic group to react and prevented gelation during the reaction for introducing a (meth)acrylic group and the storage of compounds.

As a stabilizer, for example, sulfur-containing type stabilizers are known for an urethane(meth)acrylate oligomer (Patent Document 3), a butadiene rubber (Patent Document 4) or a thermoplastic elastomer (Patent Document 5).

### PRIOR ART DOCUMENTS

### Patent Documents

Patent Document 1: Japanese Unexamined Patent Application Publication No. 2006-045284
Patent Document 2: Japanese Unexamined Patent Application Publication No. 2007-211240
Patent Document 3: Japanese Unexamined Patent Application Publication No. 2001-316434
Patent Document 4: Japanese Unexamined Patent Application Publication (Translation of PCT Application) No. 2003-535926
Patent Document 5: Japanese Unexamined Patent Application Publication No. 2008-075058

### SUMMARY OF THE INVENTION

### Object to be Solved by the Invention

However, those stabilizers have been unknown that inhibit gelation during production and storage of a (hydrogenated) polybutadiene derivative having a (meth)acryl group and that does not result in the coloring of a cured product.

An object of the present invention is to provide a stabilizer that acts as a polymerization inhibitor during production and storage of a (meth)acrylic modified (hydrogenated) polybutadiene and does not result in yellowing of a cured product.

### Means to Solve the Object

The present inventors have made a keen study to solve the object and have found that, by adding a hindered phenol based stabilizer having a thioether group, polymerization is prevented and yellowing upon curing does not occur. The present invention is thus completed.

The present invention relates to: (1) a curable composition containing 0.1-99.9999% by weight of a component (A) and 0.0001-10% by weight of a component (B), wherein the component (A) is a (meth)acrylic modified (hydrogenated) polybutadiene polymer represented by formula (I)

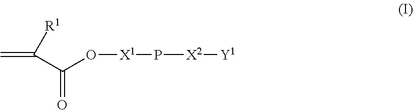

[wherein P represents a polymer having 0-100 mol % of a repetition unit represented by formula (II)

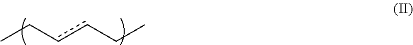

(wherein the solid and dotted double line part represents a single bond or a double bond) and 100-0 mol % of a repetition unit represented by formula (III)

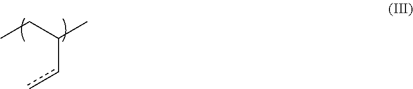

(wherein the solid and dotted double line part represents a single bond or a double bond), and wherein X¹ and X² each independently represent a C₁-C₂₀ linking group which may comprise an oxygen atom and/or a nitrogen atom; Y¹ represents a hydrogen atom, hydroxyl group, carboxyl group or (meth)acryloyloxy group; and R¹ represents a hydrogen atom or methyl group], and wherein the component (B) is a compound represented by formula (IV)

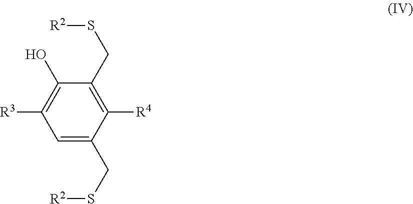

[wherein each R² independently represents a C₁-C₂₀ alkyl group which may have a substituent, a phenyl group which may have a substituent or a benzyl group which may have a substituent;
R³ and R⁴ each independently represent a C₁-C₁₀ alkyl group which may have a substituent, or a C₅-C₁₂ cycloalkyl group which may have a substituent],
(2) the curable composition according to (1), wherein the (meth) acrylic modified (hydrogenated) polybutadiene polymer is a polymer wherein the repetition unit represented by formula (III) is 85 mol % or more,
(3) the curable composition according to (1) or (2), wherein a dispersion degree of the (meth)acrylic modified (hydrogenated) polybutadiene polymer is within a range from 1.01 to 2.00, and
(4) the curable composition according to any one of (1) to (3), wherein the (meth)acrylic modified (hydrogenated) polybutadiene polymer is a polymer wherein a (meth)acrylic acid group is introduced in 80 mol % or more of all the hydroxyl groups in a polymer represented by formula (V)

HO—X¹—P—X²—Y² (V)

(wherein X¹, P and X² have the same meaning as defined above; and Y² represents a hydrogen atom, hydroxyl group or carboxyl group).

The present invention further relates to (5) a cured product which is provided by curing the curable composition according to any one of (1) to (4).

### MODE OF CARRYING OUT THE INVENTION

A (meth)acrylic modified (hydrogenated) polybutadiene of the present invention is not particularly limited as long as it has a (meth)acryloyloxy group at least at one of the terminals. In the present invention, (meth)acrylic acid means acrylic acid or methacrylic acid. Further, a (hydrogenated) polybutadiene means polybutadiene or a hydrogenated product thereof.

(Compounds Represented by Formula I))

In formula (I), X¹ and X² respectively represent a C₁-C₂₀ linking group which may comprise an oxygen atom and/or a nitrogen atom.

Examples of the C₁-C₂₀ linking group which may comprise an oxygen atom and/or a nitrogen atom include a divalent straight or branched chain C₁-C₂₀ alkylene group, a divalent straight or branched chain C₂-C₂₀ alkylene group having an ether bond, and a group represented by formula (VI)

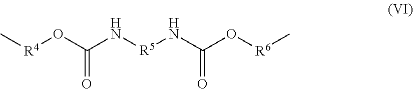

(in formula (VI), R⁴ to R⁶ each independently represent a divalent straight or branched chain C₁-C₁₀ alkylene group, a C₃-C₈ cycloalkylene group which may have a C₁-C₆ alkyl group as a substituent, a C₅-C₈ aromatic group which may have a C₁-C₆ alkyl group as a substituent, or a group combining the above-mentioned groups).

Specifically, examples of the divalent straight or branched chain C₁-C₂₀ alkylene group include methylene, ethylene, propylene, methylethylene, butylene, 1,2-dimethylethylene, pentylene, 1-methylbutylene, 2-methylbutylene hexaethylene heptaethylene octaethylene, nonaethylene and decaethylene. Examples of the divalent straight or branched chain C₂-C₂₀ alkylene group having an ether bond include —(CH₂O)ₐ(CH₂)— [“a” represents an integer of 1 to 17], —(CH₂CH₂O)_{b}(CH₂CH₂)— [“b” represents an integer of 2 to 8] and —(CH₂CH₂CH₂O)_{c}(CH₂CH₂CH₂)— [“c” represents an integer of 1 to 5].

Examples of the divalent straight or branched chain C₁-C₁₀ alkylene group in formula (VI) include those that are the same as the specific examples for formula (I). Examples of the C₃-C₈ cycloalkylene group which may have a C₁-C₈ alkyl group as a substituent include cyclopropylene, 2-methylcyclopropylene, cyclobutylene, 2,2-dimethylcyclobutylene, cyclopentylene, 2,3-dimethylcyclopentylene, cyclohexylene, 1,3,3-trimethylcyclohexylene and cyclooctylene. Examples of the C₅-C₈ aromatic group which may have a C₁-C₆ alkyl group as a substituent include 1,4-phenylene and 2-methyl-1,4-phenylene.

Examples of the group combining the above-mentioned groups include methylene-cyclopropylene, methylene-cyclopentylene, methylene-2,3-dimethylcyclopentylene, methylene-1,3,3,-trimethylcyclohexylene, ethylene-cyclopropylene, ethylene-cyclohexylene, ethylene-3,3-dimethylcyclohexylene, methylene-cyclopropylene-methylene, ethylene-cyclohexylene-methylene, and hexylene-cyclohexylene-methylene. The order in a combined group may be switched.

Examples of formula (VI) include:

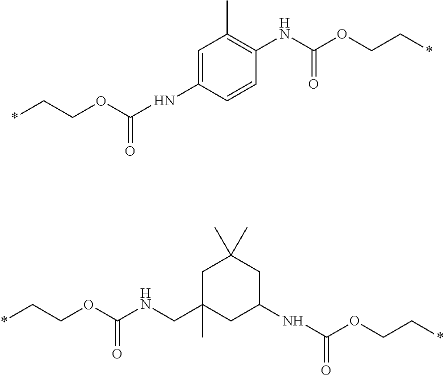

(“*” represents positions of connection)

“P” in formula (I) is a polybutadiene chain or a hydrogenated polybutadiene chain having formula (II) and/or formula (III) as a repetition unit. When the solid and dotted double line part is a double bond, it represents a non-hydrogenated polybutadiene, and when the solid and dotted double line part is a single bond, it represents a hydrogenated polybutadiene.

Further, when the 1,4-bond repetition unit represented by formula (II) has a double bond, a trans structure, a cis structure, or a trans and cis mixture structure can be present.

Ratios of the 1,4-bond repetition unit represented by formula (II) and the 1,2-bond repetition unit represented by formula (III) are respectively 0-100 mol %. However, according to the measurement by Morello method, the 1,2-bond repetition unit represented by formula (III) are preferably present by 80% or more, more preferably 85% or more, further preferably 90% or more and particularly preferably 95% or more.

In formula (I), Y¹ may be unsubstituted (hydrogen atom) or may have a substituent. Examples of the substituent include a hydroxyl group, carboxyl group and (meth)acryloyloxy group, wherein a (meth)acryloyloxy group is preferred.

The number average molecular weight of a (meth)acrylic)acrylic modified (hydrogenated) polybutadiene of the present invention is usually about 500 to 10000 and preferably 1000 to 5000.

Value obtained by dividing the weight average molecular weight of a polymer by the number average molecular weight represents a dispersion degree. The smaller the value of dispersion degree is, the narrower the dispersion is, and in that case the polymers are constituted by ones with relatively similar molecular weights. A polymer entirely constituted by the same molecular weights has a dispersion degree of 1. Further, the larger the dispersion degree is, the broader the dispersion is. Thus, the polymers are constituted by a mixture of lower to higher molecular weight polymers, thereby resulting in a weak strength of a cured product upon curing or in abroad range of curing temperature. For this reason, a smaller dispersion degree is preferred to obtain good polymers. Dispersion degree of a (meth)acrylic modified (hydrogenated) polybutadiene of the present invention is 1.01 to 2.00, preferably 1.01 to 1.50, and further preferably 1.01 to 1.30.

A hydrogenated polybutadiene is produced by reducing the double bond of polybutadiene with hydrogen. Here, while the hydrogenation ratio is not particularly limited, a hydrogenation ratio of 90% or more is preferred, 99% or more is more preferred, and 99.5% or more is still more preferred. Remaining double bonds can be subjected to a quantification analysis employing an iodine addition reaction (hereinafter referred to as “iodine value”), wherein the iodine value is 100 or less, preferably 50 or less, more preferably 25 or less, and still more preferably 15 or less.

The (meth)acryl group introduction ratio in a (meth)acrylic modified (hydrogenated) polybutadiene is a percentage value of the introduction ratio of (meth)acryl groups relative to all the hydroxyl groups before the introduction of (meth)acryl groups. The (meth)acryl group introduction ratio in a (meth)acrylic modified hydrogenated polybutadiene of the present invention is 80% or more, preferably 90% or more, and further preferably 95% or more.

(Acryl Group Introducing Reaction)

The method of producing a (meth)acrylic modified (hydrogenated) polybutadiene comprises reacting a compound comprising (meth)acryl group with a hydroxyl group of a hydroxyl group-containing polybutadiene or a hydroxyl group-containing hydrogenated polybutadiene to conduct the introduction of (meth)acryl groups, but the method is not limited to any particular method.

Specifically, examples of the compound comprising (meth)acryl group include (meth)acrylic acids such as acrylic acid and methacrylic acid; (meth)acrylic acid esters such as methyl acrylate, methyl methacrylate, ethyl acrylate and ethyl methacrylate; and a hydroxyl group-containing (meth)acrylic acid esters such as 2-hydroxyethyl acrylate and 2-hydroxyethyl methacrylate.

Further, those methods known as a (meth)acryl group introducing method include a method comprising dehydration and condensation of (meth)acrylic acids and a hydroxyl group of polybutadiene using such as p-toluene sulfonic acid as a catalyst; a method comprising introducing a (meth)acryl group by interesterification of (meth)acrylic acid esters and a hydroxyl group of polybutadiene using a titanium catalyst, a tin catalyst, etc. as a catalyst; and a method comprising reacting a compound having 2 or more isocyanate groups and a hydroxyl group-containing (meth)acrylic acid esters such as a acrylic acid 2-hydroxyester and a hydroxyl group of polybutadiene.

These reactions are conducted at or higher than room temperature, whereby a polymerization reaction, etc. is caused during the reaction and results in the appearance of high-molecular weight peaks in GPC besides that of the target. In addition, the product may tend to get a higher viscosity than that of the target. Products to which (meth)acryl groups are introduced are stored in a container, and even if the temperature is lower than the reaction temperature, a high-molecular weight peak gradually tends to appear or the product tends to get a higher viscosity. These events are due to a polymerization reaction by (meth)acrylic acid groups and can be prevented by admixing a stabilizer.

(Compounds Represented by Formula (IV))

In formula (IV), each R² independently represents a C₁-C₂₀ alkyl group which may have a substituent, a phenyl group which may have a substituent or a benzyl group which may have a substituent, and R³ and R⁴ each independently represent a C₁-C₁₀ alkyl group which may have a substituent, or a C₅-C₁₂ cycloalkyl group which may have a substituent.

Examples of the C₁-C₂₀ alkyl group for R² include a methyl group, ethyl group, n-propyl group, isopropyl group, n-butyl group, sec-butyl group, t-butyl group, isobutyl group, n-pentyl group, isopentyl group, neopentyl group, t-pentyl group, n-hexyl group, isohexyl group, 1-methylpentyl group, 2-methylpentyl group, n-heptyl group, n-octyl group, n-nothyl group, n-decyl group, n-undecyl group, n-dodecyl group, n-tridecyl group, n-tetradecyl group, n-pentadecyl group, n-hexadecyl group, n-heptadecyl group, n-octadecyl group, n-nonadecyl group and n-icosyl group.

Examples of the substituent for a C₁-C₂₀ alkyl group which may have a substituent, a phenyl group which may have a substituent or a benzyl group which may have a substituent specifically include a hydroxyl group; a halogen atom such as a fluorine atom, chlorine atom, bromine atom and iodine atom; a C₁-C₆ alkyl group such as a methyl group, ethyl group, n-propyl group, isopropyl group, n-butyl group, sec-butyl group, t-butyl group, n-pentyl group, isopentyl group, neopentyl group, t-pentyl group, n-hexyl group, isohexyl group, 1-methylpentyl group and 2-methylpentyl group; a C₁-C₆alkoxy group such as a methoxy group, ethoxy group, n-propoxy group, isopropoxy group, n-butoxy group, sec-butoxy group and t-butoxy group; a —OCOR⁷ group; a —COOR⁸ group; and a —CONR⁹R¹⁰ group.

R⁷ and R⁸ each independently represent a C₁-C₂₀ alkyl group or a C₂-C₈ alkenyl group, and R⁹ and R¹⁰ each independently represent hydrogen, a C₁-C₂₀ alkyl group or a C₂-C₈ alkenyl group.

Specific examples of the C₁-C₂₀ alkyl group include those groups that are the same as the specific examples for R². Examples of the C₂-C₈ alkenyl group include a vinyl group, allyl group, isopropenyl group, 1-propenyl group, 2-propenyl group, 1-butenyl group, 2-butenyl group, 3-butenyl group, 1,3-butanedienyl group and 2-methyl-2-propenyl group.

In formula (IV), a C₁-C₁₀ alkyl group for R³ is specifically exemplified by those alkyl groups that meet the requirement of C₁-C₁₀ among the specific examples for R².

Examples of the C₃-C₁₂ cycloalkyl group include cyclopropyl, cyclobutyl, cyclopentyl, cyclohexyl, cyclooctyl and cyclodecyl.

Examples of the C₁-C₁₀ alkyl group which may have a substituent and the C₃-C₁₂ cycloalkyl group which may have a substituent specifically include a hydroxyl group; a halogen atom such as a fluorine atom, chlorine atom, bromine atom and iodine atom; a C₁-C₆ alkyl group such as a methyl group, ethyl group, n-propyl group, isopropyl group, n-butyl group, sec-butyl group, t-butyl group, n-pentyl group, isopentyl group, neopentyl group, t-pentyl group, n-hexyl group, isohexyl group, 1-methylpentyl group and 2-methylpentyl group; and a C₁-C₆ alkoxy group such as a methoxy group, ethoxy group, n-propoxy group, isopropoxy group, n-butoxy group, sec-butoxy group and t-butoxy group,

Examples of formula (IV) include 4,6-bis(octylthiomethyl)-o-cresol and 4,6-bis(dodecylthiomethyl)-o-cresol.

While a compound represented by formula (IV) may be admixed either before initiating a reaction to introduce an acryl group or after the reaction has been initiated, it is preferred to admix the compound before initiating a reaction.

(Curable Composition and Cured Product)

As for a curable composition of the present invention, while the content of a (meth)acrylic modified (hydrogenated) polybutadiene represented by formula (I) is not particularly limited, the content is preferably 0.1-99.9999% by weight, preferably 1-99.9% by weight and preferably 10-99% by weight. Further, the content of a compound represented by formula (IV) is not particularly limited, but an amount at least sufficient to prevent polymerization is necessary, and it is necessary that the amount is equal to or less than the amount that inhibits curing reaction to occur when the curing reaction is taking place. Specifically, the amount of a compound represented by formula (IV) is preferably 0.0001-10% by weight, preferably 0.001-5% by weight, and preferably 0.01-1% by weight.

In the present invention, a composition in which other components are added to cure a (meth)acrylic modified (hydrogenated) polybutadiene is referred to as a curable composition, and a substance obtained by curing the curable composition is referred to as a cured product.

A (meth)acrylic modified (hydrogenated) polybutadiene of the present invention can be cured with heat, light, a radical polymerization initiator, etc. along with other additives, depending on the purpose. Alternatively, the (meth)acrylic modified (hydrogenated) polybutadiene may be cured by Michael addition method.

The heating method is not particularly limited and conventionally known heating methods such as a heater may be employed.

As for light, while, for example, ultraviolet, visible light, X-ray and electron beam can be used, it is preferred to use ultraviolet. Due to high energy of ultraviolet, curing reaction can be accelerated by irradiating ultraviolet to a curable composition, which results in acceleration of the curing rate of a curable composition as well as in reduction of the amount of an unreacted curable composition in the cured product.

The method of irradiating visible light is not particularly limited and those using such as an incandescent lamp and a fluorescent light are exemplified. Further, the means of irradiating ultraviolet is not particularly limited, where the examples for the electrode system include a metal halide lamp, xenon lamp, low-pressure mercury lamp, high-pressure mercury lamp and ultrahigh-pressure mercury lamp, and the examples for the electrodeless system include an excimer lamp and a metal halide lamp. While the range of wavelength is not particularly limited when using ultraviolet, a range of 150-400 nm is preferred and 200-380 nm is more preferred. As for the atmosphere for irradiating ultraviolet, although an inactive gas atmosphere such as nitrogen gas and carbon dioxide gas or an atmosphere in which oxygen concentration is decreased is preferred, normal air atmosphere is also possibly used. Temperature of the irradiation atmosphere can usually be 10-200° C.

Since curing status can be measured using such as a Fourier transform infrared spectroscopic analysis device and a photochemical reaction calorimeter, curing conditions (light irradiation time, light intensity, heating temperature, heating time, etc.) for a cured product to be completely cured can by selected appropriately.

Although curing reaction can be conducted with only a (meth)acrylic modified (hydrogenated) polybutadiene of the present invention, it can also be conducted by adding a polymerizable vinyl compound.

Examples of the polymerizable vinyl compound include an aromatic vinyl compound such as styrene, vinyltoluene, α-methylstyrene and divinylbenzene; unsaturated carboxylic acid esters such as methyl (meth)acrylate, 2-hydroxyethyl (meth)acrylate, t-butyl (meth)acrylate, n-hexyl (meth)acrylte, isobutyl (meth)acrylate, cyclohexyl (meth)acrylate, 2-ethylhexyl (meth)acrylte, lauryl (meth)acryate, stearyl (meth)acrylate, benzyl (meth)acrylte, mono- or di(meth)acrylate of (poly)ethyleneglycol, mono- or di(meth)acrylate of (poly)propyleneglycol, mono- or di(meth)acrylate of 1,4-butanediol, and mono-, di- or tri(meth)acrylate of trimethylolpropane; an allylated product such as diallyl phthalate, diallyl acrylamide, triallyl (iso)cyanurate,triallyltrimellitate, o,o′-diallylbisphenol A, o,o′-diallylbisphenol F, 1,1,1,3,3,3-hexafluoro-2,2-bis(p-hydroxy-o-allylphenyl)pro pane, allylated phenolnovolac, 1,1,3-tris-(4-hydroxyphenyl)propane, 1,1,2,2-tetra(4-hydroxyphenyl)ethane, a dehydrated condensate of phenols and hydroxybenzaldehyde; (poly)oxyalkyleneglycol di(meth)acrylate such as (poly)ethyleneglycol di(meth)acrylate and (poly)propyleneglycol di(meth)acrylate; and a terminal acrylic-modified polybutadiene such as TEA-1000, TE-1000 and TEAI-1000 (manufactured by NIPPON SODA CO., LTD.). Further exemplified are conjugated diene compounds such as butadiene, isoprene and chloroprene; and compounds containing a reactive functional group such as acrylic acid, methacrylic acid, itaconic acid, fumaric acid, glycidyl methacrylate, vinylpyridine, diethylaminoethyl acrylate, N-methyl methacrylamide and acrylonitrile. These polymerizable vinyl compounds may be used alone or as a mixture of two or more kinds.

A radical polymerization initiator is a compound that releases a substance which initiates a radical polymerization by light irradiation and/or heating. Examples of the radical polymerization initiator include an organic peroxide, imidazole derivative, bisimidazole derivative, N-arylglycin derivative, organic azide compound, titanocenes, aluminate complex, N-alkoxypyridinium salt and thioxanthone derivative. Specific examples of the organic peroxide include hydroperoxides such as t-butylhydroperoxide, p-menthane hydroperoxide, cumene hydroperoxide and diisopropylbenzene hydroperoxide; peroxyesters such as t-butylperoxylaurate, t-butylperoxybenzoate, and t-butylperoxydecanoate; peroxyketals such as 1,5-di-t-butylperoxy-3,3,5-trimethylcyclohexane; ketoneperoxides such as ethyl peroxyacetoacetate; and diacylperoxides such as benzoyl peroxide. Examples of the radical polymerization initiator further include benzoin, benzoinisopropylether, benzoinisobutylether, 2,2-diethoxyacetophenone, 2,2-dimethoxyphenylacetophenone, 2-ethylanthraquinone, 1,3-di(tert-butyldioxycarbonyl)benzophenone, 4,4′-tetrakis(tert-butyldioxycarbonyl)benzophenone, 3-phenyl-5-isoxazolone, 2-mercaptobenzimidazole, bis(2,4,5-triphenyl)imidazol, 2,2-dimethoxy-1,2-diphenylethane-1-on (product name: IRGACURE® 651, manufactured by Ciba Specialty Chemicals Inc.), 1-hydroxy-cyclohexyl-phenyl-ketone (product name: IRGACURE® 184, manufactured by Ciba Specialty Chemicals Inc.), 2-benzyl-2-dimethylamino-1-(4-morpholinophenyl)-butane-1-o n (product name: IRGACURE® 369, manufactured by Ciba Specialty Chemicals Inc.), bis(η⁵-2,4-cyclopentadiene-1-yl)-bis(2,6-difluoro-3-(1H-py rrole-1-yl)-phenyl)titanium) (product name: IRGACURE® 784, manufactured by Ciba Specialty Chemicals Inc.), dicumyl peroxide (DCP), t-butylperbenzoate (TBPB) and t-butylperoxy hexyne-3. These radical polymerization initiators may be used alone or as a mixture of two or more kinds.

A (meth)acrylic modified (hydrogenated) polybutadiene obtained by the production method of the present invention can be produced as an unclouded transparent liquid so that it is also useful as an adhesive or a paint for goods such as optical devices for which transparency is required, and is also useful as high-frequency polymeric materials such as electronic materials and antenna materials owing to its low induction ratio.

### EXAMPLES

A recording material of the present invention is explained in detail below with reference to Examples, but the present invention shall not necessarily be limited to those Examples.

### 1. Production of an Acrylic Modified (Hydrogenated) Polybutadiene

### Production Example 1

In a 500 mL glass reaction vessel, 300 g of a hydroxyl group-containing polybutadiene (product name: G-3000, manufactured by NIPPON SODA CO., LTD.), 0.14 g of 4,6-bis(octylthiomethyl)-o-cresol (product name: HP-400, manufactured by Kawaguchi Chemical Industry Co., LTD.), 79 g of ethyl acrylate, and 1.73 g of dioctyl tin dilaurate were prepared and reacted for 5 hours at about 120° C. to obtain an acrylic modified polybutadiene. It was confirmed by GPC that no high-molecular weight structures were generated other than the target.

### Production Example 2

An acrylic modified polybutadiene was obtained by the method described in Production Example 1 except that 4,6-bis(dodecylthiomethyl)-o-cresol (product name: IRGANOX® 1726, manufactured by Ciba Specialty Chemicals Inc.) was used in place of 4,6-bis(octylthiomethyl)-o-cresol used in Production Example 1.

### Production Example 3

In a 3 L glass reaction vessel, 1500 g of a hydroxyl group-containing hydrogenated polybutadiene (product name: GI-1000, manufactured by NIPPON SODA CO., LTD.), 3.9 g of 4,6-bis(octylthiomethyl)-o-cresol (product name: HP-400, manufactured by Kawaguchi Chemical Industry Co., LTD.), 397 g of ethyl acrylate, and 8.62 g of dioctyl tin dilaurate were prepared and reacted for 5 hours at about 120° C. to obtain an acrylic modified hydrogenated polybutadiene. It was confirmed by GPC that no high-molecular weight structures were generated other than the target.

### Comparative Production Example 1

An acrylic modified polybutadiene was obtained by the method described in Production Example 1 except that 2,6-di-t-butyl-4-hydroxytoluene (abbreviation: BHT) was used in place of 4,6-bis(octylthiomethyl)-o-cresol used in Production Example 1.

### Comparative Production Example 2

An acrylic modified polybutadiene was obtained by the method described in Production Example 3 except that 2,6-di-t-butyl-4-hydroxytoluene (abbreviation: BHT) was used in place of 4,6-bis(octylthiomethyl)-o-cresol used in Production Example 3.

### Heat Stability Test

### Test Example 1

Resin obtained in Production Example 3 was placed in an oven of 150° C. Then, the coloring status and the status of increase in high-molecular weight structures observed by GPC were confirmed. The status after 4 and 10 days are shown in Table 1.

### Test Example 2

The coloring status and the status of increase in high-molecular weight structures were confirmed by the method described in Test Example 1 except that the resin obtained in Comparative Production Example 2 was used in place of the resin obtained in Production Example 3.

TABLE 1
Result of heat stability test

| | Acrylic | | |
|---|---|---|---|
| | modified | | Status in heat stability |
| | polybutadiene | Stabilizer | test |
| Production | Acrylic | HP-400 | 4 days after: colorless |
| Example 3 | modified | | 10 days after: colorless, no |
| | GI-1000 | | increase in high-molecular |
| | | | weight structures |
| Comparative | Acrylic | BHT | 4 days after: yellow |
| Production | modified | | 10 days after: yellow, |
| Example 2 | GI-1000 | | gelated |

Comparison of Production Example and Comparative Production Example 2 demonstrated that a superior storage stability was obtained and no coloring occurred when using the stabilizer of the present invention.

### 2. Production of Curable Composition and Cured Product

### Example 1)

A curable composition was obtained by the addition of 2 g of the resin obtained in Production Example 1, 18 g of methyl methacrylate and 0.25 g of benzoylperoxide. This curable composition was subjected to curing for 6 hours at 70° C. and for further 2 hours at 100° C. to obtain a cured product. The post-curing status is shown in Table 2.

### Example 2

A cured product was obtained by the method described in Example 1 except that the resin obtained in Production Example 2 was used in place of the resin obtained in Production Example 1 used in Example 1. The post-curing status is shown in Table 2.

### Comparative Example 1

A cured product was obtained by the method described in Example 1 except that the resin obtained in Comparative Production Example 1 was used in place of the resin obtained in Production Example 1 used in Example 1. The post-curing status is shown in Table 2.

TABLE 2
Confirmation of stability status

| | | | Coloring |
|---|---|---|---|
| | | | status of |
| Production | Acrylic modified | | cured |
| Example | polybutadiene | Stabilizer | product |
| Example 1 | Acrylic modified | HP-400 | Colorless |
| | G-3000 | | |
| Example 2 | Acrylic modified | IRGANOX1726 | Colorless |
| | G-3000 | | |
| Comparative | Acrylic modified | BHT | Yellow |
| Example 1 | G-3000 | | |

Comparison of Examples 1 and 2 to Comparative Example 1 showed that coloring does not occur when using the stabilizer of the present invention.

### INDUSTRIAL APPLICABILITY

By admixing a compound represented by formula (IV), those effects are provided that gelation during production and storage of a (hydrogenated) polybutadiene derivative having a (meth)acrylic group is inhibited and further that coloring does not occur upon curing. No other stabilizer exerts these effects.

1. A curable composition containing 0.1-99.9999% by weight of a component (A) and 0.0001-10% by weight of a component (B), wherein the component (A) is a (meth)acrylic modified (hydrogenated) polybutadiene polymer represented by formula (I) 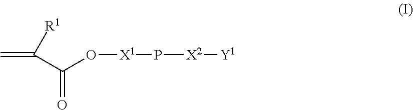
[wherein P represents a polymer having 0-100 mol % of a repetition unit represented by formula (II) 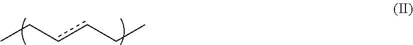
(wherein the solid and dotted double line part represents a single bond or a double bond) and 100-0 mol % of a repetition unit represented by formula (III) 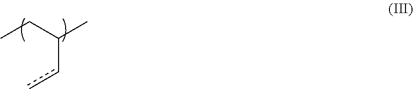
(wherein the solid and dotted double line part represents a single bond or a double bond), and wherein X¹ and X² each independently represent a C₁-C₂₀ linking group which may comprise an oxygen atom and/or a nitrogen atom; Y¹ represents a hydrogen atom, hydroxyl group, carboxyl group or (meth)acryloyloxy group; and R¹ represents a hydrogen atom or methyl group], and wherein the component (13) is a compound represented by formula (IV) 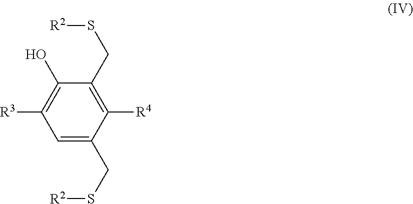
[wherein each R² independently represents a C₁-C₂₀ alkyl group which may have a substituent, a phenyl group which may have a substituent or a benzyl group which may have a substituent; R³ and R⁴ each independently represent a C₁-C₁₀ alkyl group which may have a substituent, or a C₅-C₁₂ cycloalkyl group which may have a substituent].

2. The curable composition according to claim 1, wherein the(meth)acrylic modified (hydrogenated) polybutadiene polymer is a polymer wherein the repetition unit represented by formula (III) is 85 mol % or more.

3. The curable composition according to claim 1, wherein a dispersion degree of the (meth)acrylic modified (hydrogenated) polybutadiene polymer is within a range from 1.01 to 2.00.

4. The curable composition according to claim 1, wherein the (meth)acrylic modified (hydrogenated) polybutadiene polymer is a polymer wherein a (meth)acrylic acid group is introduced in 80 mol % or more of all the hydroxyl groups in a polymer represented by formula (V)
HO—X¹—P—X²—Y² (V)
(wherein X¹, P and X² have the same meaning as defined above; and Y² represents a hydrogen atom, hydroxyl group or carboxyl group).

5. A cured product which is provided by curing the curable composition according to claim 1.
